(19) United States
(12) Patent Application Publication
(10) Pub. No.: US 2007/0029259 A1
(43) Pub. Date: Feb. 8, 2007

(54) Method of reducing impurity content in aqueous salt solution

(76) Inventors: Hirotaka Kakita, Takamatsu-shi (JP); Akinari Sonoda, Takamatsu-shi (JP); Kazutoshi Yoshihara, Takamatsu-shi (JP); Hiroshi Kamishima, Takamatsu-shi (JP); Takahiro Hirotsu, Takamatsu-shi (JP); Kenta Ooi, Takamatsu-shi (JP); Kazuyuki Takashima, Sakaide-shi (JP); Tetuo Iwasaki, Sakaide-shi (JP); Eiji Santoki, Sakaide-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/543,530

(22) PCT Filed: Feb. 4, 2004
(86) PCT No.: PCT/JP04/01140

§ 371 Date: Aug. 15, 2006

(30) Foreign Application Priority Data
Feb. 4, 2003 (JP) 2003-027693
Feb. 4, 2003 (JP) 2003-027694
Feb. 4, 2003 (JP) 2003-027691
Feb. 4, 2003 (JP) 2003-027692

(51) Int. Cl. C02F 1/42

(52) U.S. Cl. 210/660

(57) ABSTRACT

A method in which an aqueous water-soluble compound solution containing at least one impurity in a slight amount is passed through a column packed with an adsorbent onto which the impurity is selectively adsorbed to thereby remove the impurity from the aqueous solution, wherein an abnormal phenomenon in adsorption chromatography is caused to obtain an elute fraction having a higher concentration of the trace impurity than the raw solution and this fraction is removed. Thus, the trace impurity can be efficiently removed and the water-soluble compound can be obtained in a high-purity state. Applying this method to aqueous sodium chloride solutions gives sodium chloride crystals reduced in impurity concentration. Dissolving such sodium chloride crystals, which have a low potassium ion concentration, in water provides an aqueous sodium chloride solution for medical use. Also provided is a sodium chloride composition for preparing an artificial seawater for use in alga cultivation which is reduced in magnesium ion or calcium ion concentration.

### FIELD OF THE INVENTION

The present invention relates to a method of removing trace impurities or, particularly, impurity ions contained in an aqueous solution in which specified materials such as water-soluble inorganic salts are dissolved by utilizing a phenomenon of abnormal adsorption chromatography; a method of producing purified sodium chloride crystals having a purity of 99.99% by mass or higher by utilizing the above method; low potassium medical saline which is produced by dissolving purified sodium chloride crystals reduced in the content of potassium ions and is inhibited in functional disorders caused by potassium ions and a method of producing this saline as well as a sodium chloride composition for preparing artificial seawater for culturing of algae by dissolving it in water according to need prior to its use and a method of producing the composition.

### BACKGROUND OF THE INVENTION

As a method of separating each component in a mixture containing two or more types of components, a distillation method, recrystallization method, solvent extraction method and non-solvent washing method and the like are known. Attention has been focused in recent years to chromatography (hereinafter referred to simply as chromato) in industrial fields because each component can be separated in a high purity by a relatively simple procedure.

This chromatography is carried out by passing a solution in which a mixture to be separated in a desired solvent, through a column filled with a solid adsorbent. In this method, each component is isolated by utilizing a phenomenon, namely, the so-called phenomenon of adsorption chromatography, that each component is made to have a different moving speed because each component in the solution is distributed in a different proportion between the solid adsorbent side (stationary phase) and the traveling liquid side (mobile phase).

This method for continuously obtaining a fraction of each concentrated component by utilizing such a phenomenon of adsorption chromatography is widely practiced industrially in various fields. Examples of the methods proposed heretofore as this method include a method in which a mixture of several components and a desorbing agent are alternately introduced into a column filled with an adsorbent such that the adsorption zone of each component is overlapping on a part of the adjacent adsorption zone of another component under control (the publication of Japanese Patent Kokai (JP-A) No. 57-207507) and a method in which a fraction containing the desired components and excluding a desorbing agent and a starting material mixture are supplied consecutively to the column (the publication of JP-A No. 58-20208).

In the meantime, the selectivity of an adsorbent is an important factor in the phenomenon of adsorption chromatography and various adsorbents are therefore developed corresponding to each object. For example, an adsorbent obtained by extracting a black manganese oxide hydrate with an acid (the publication of JP-A No. 8-38887) and a binder-free 3A-type zeolite beads adsorbent having specific properties (the publication of JP-A No. 2002-119849) are proposed as an adsorbent that adsorbs potassium selectively.

However, many impurities contained in trace amounts in an aqueous solution generally have properties similar to those of the principal component. Therefore, with regard to adsorbents, the coefficient of selection of impurities from the principal component is small and also there is a theoretical limitation to an improvement in this selection coefficient. It is therefore a very difficult matter to separate impurities from the principal component.

Specifically, in the phenomenon of ordinary adsorption chromatography, an adsorbent exhibiting selectivity to a specified component in a mixture solution is used as the stationary phase and the mixture solution as the mobile phase and the mixture solution is made to flow through a column filled with an adsorbent to fractionate the flowing fractions with time, thereby collecting a fraction containing the concentrated specified component. As to the concentration of the specified component in the fraction at this time, this concentration never exceeds the concentration of the component in the starting solution (hereinafter referred to as an initial concentration). The adsorbent continues adsorption until the amount of the component reaches the adsorption amount determined by the adsorption capacity and volume of the adsorbent. The concentration of the component in the solution gradually decreases, the amount of adsorption is saturated and the adsorbent loses the adsorbing ability finally, with the result that the concentration of the component in the solution becomes constant.

The first embodiment according to the present invention has been attained with an intention to remove a trace amount of impurities, especially, impurity ions present in an aqueous solution of a water-soluble compound efficiently by utilizing the phenomenon of adsorption chromatography quite different from the foregoing phenomenon of ordinary adsorption chromatography to yield the water-soluble compound in a high-purity state.

Next, as a method of obtaining sodium chloride crystals from a dilute sodium chloride solution such as seawater or salt water, the following methods are known so far: a method in which particles of limestone, marble or calcite are brought into contact with the above solution to react (the publication of JP-A No. 53-91151), a method in which flame is blown into a drum body of a rotary drum type drier and seawater is sprayed onto the outside surface of the drum body to evaporate water to thereby deposit crystals (the publication of JP-A No. 2000-228964 and a method in which seawater is supplied in an inside water tank, hot air is blown against the surface of water in the inside water tank and at the same time, the seawater in the inside water tank is heated by hot water contained in an outside water tank disposed on the outside of the inside water tank to evaporate water, thereby making salt (the publication of JP-A No. 2001-158616).

However, these sodium chloride crystals obtained in this manner contain potassium ions, magnesium ions and calcium ions as impurities and it is therefore necessary to purify these crystals to upgrade their purities when they are used as an industrial material which need high purity.

As methods of purifying such crude sodium chloride, the following methods have been proposed so far: a method in which bay salt particles are screened under fluidization of them in saturated brine to remove a part containing particles having smaller diameters and containing a large amount of impurities (the publication of JP-A No. 8-119627) and a method in which a calcium compound is added to sodium chloride brine contaminated with potassium chloride and sulfate ions to precipitate and remove these sulfate ions as calcium sulfate and then sodium chloride crystals are collected from the residual solution by precipitation (the publication of Japanese Patent Kokai No. 2002-523330).

On the other hand, there are various methods proposed as a method of continuously obtaining fractions each having a concentrated component from a mixture containing two or more components by using a sold adsorbent and by utilizing the phenomenon of ordinary adsorption chromatography. Some of these methods have been already industrially practiced (publications of JP-A Nos. 57-207507, 57-207508 and 58-20208).

In a separating method utilizing these phenomena of adsorption chromatography, the selection of an adsorbent is important. Therefore, many inorganic ion-exchangers (publications of JP-A Nos. 3-153522, 8-38887 and 2002-119849) and chelate resins (the publication of JP-A No. 5-186215) which disclose that high coefficients of selection of desired components have been developed.

However, potassium ions, magnesium ions and calcium ions contained usually as impurities in sodium chloride each have properties similar to those of sodium ions. These ions are therefore separated from sodium ions in a solution with difficulty and also enter the crystal lattices of sodium chloride crystals or firmly adsorb to the surface of crystals when crystallized, and are therefore not removed simply.

Therefore, the purity of a purified product is necessarily limited in the case of purifying sodium chloride crystals by utilizing the phenomenon of ordinary adsorption chromatography.

The second embodiment according to the present invention has been attained, under the circumstances, with the intention to provide a method of obtaining purified sodium chloride crystals having a purity of at least 98% by mass or, particularly, at least 99.99% by mass by utilizing a method of removing a trace amount of impurity ions contained in an aqueous solution of a water-soluble compound which method is the first embodiment of the present invention and, specifically, by utilizing, instead of the phenomenon of conventional adsorption chromatography, the phenomenon of abnormal chromatography having a behavior quite different from that of the phenomenon of conventional adsorption chromatography.

To mention the third embodiment of the present invention, common salt, i.e. sodium chloride, is used as a supplement used for recovery from various symptoms caused by a deficiency of Na⁺ or Cl⁻, isotonic solutions to body fluids for dissolving injections and physiological solutions such as a Ringer solution and a Locke's solution and also widely used in a variety of medical fields such as washing of skins, wounds and membrane mucosa, epithem and washing of medical instruments.

Incidentally, common salt is produced primarily from seawater, salt water, rock salt and crude salt. However, these materials contain potassium ions as well as sodium chloride and potassium ions are inevitably intermixed in common salt to be obtained.

The potassium ions occupy a major part of intracellular cations in human body, exist in an average concentration of about 150 mEq/l and are also contained usually in a concentration of 3.5 to 5.0 mEq/l in blood serum. Although the concentration of potassium ions in blood serum is relatively low, a variation of the potassium ions greatly affects the ratio of the potassium ion concentration in the inside to that in the outside of a cell and also has a serious influence on the functions of the cell or, particularly, the functions of nerves and muscles through membrane potential. Also, the potassium ions are an essential factor in various enzymatic reactions running in the cell and play an important role in the synthesis of proteins or glycogen.

The internal concentration of the potassium ions is dependent primarily on the amount of excretion of potassium ions controlled by the kidneys. It is known that, when the blood serum potassium ion concentration is 5.0 mEq/l or larger, because of ingestion of a large amount of potassium ions, an increase in the amount of potassium ions to be transferred from the inside to the outside of the cell and a depreciation of the potassium ion-excreting function of the kidneys, this brings about the so-called hyperkalemia, causing symptoms such as flaccid paralysis of the muscles, sensory disorders of extremities, lower extremity heavy feeling and the like (Akiyuki Okubo, “Clinical Inspection Guide, '95”, Bunkodo, 1995, p.293-298).

It is therefore desirable to evade ingestion of potassium ions from the outside. In the present condition, the potassium ion content of medical salt which is commercially available currently, when prepared as a 20% by mass aqueous solution, is as high as 0.1 to 1.2 mg/l in an average and in the case of physiological saline usually used, the potassium ion content is also as high as 0.3 mg/l in an average.

Incidentally, in order to drop the concentration of potassium ions in an aqueous solution, it is the simplest method to allow the aqueous solution to pass through a column filled with an adsorbent, such as zeolite, having a capacity to adsorb inorganic ions selectively. However, because the theoretical value of the coefficient of selection of conventional zeolite for the selection of sodium ions from potassium ions is about 100, zeolites cannot be used to remove potassium ions from a high-concentration aqueous sodium chloride solution.

The third embodiment according to the present invention has been attained with the intention to provide low-potassium medical saline produced by using purified sodium chloride crystals having a reduced content of potassium ions and dissolving these crystals in water to suppress ingestion of potassium ions from outside to prevent the onset of hyperkalemia under this situation.

To mention the fourth embodiment according to the present invention, artificial seawater for culturing of algae is usually adjusted to have a composition close to that of natural seawater. And, natural seawater contains about 35 g of various inorganic salts in 1 kg of the natural seawater and the contents of major ions and compounds contained therein are as shown in Table 1 (Japan Seawater Association/Japan Salt Science Research Foundation, “Science and Industry of Seawater”, 1994, p. 28).

TABLE 1

| Kind of ion or compound | Content, g |
|---|---|
| Na⁺ | 10.5561 |
| Mg⁺⁺ | 1.2720 |
| Ca⁺⁺ | 0.4001 |
| K⁺ | 0.3800 |
| Sr⁺⁺ | 0.0133 |
| Cl⁻ | 18.9799 |
| SO₄⁻ | 2.6486 |
| HCO₃⁻ | 0.1397 |
| Br⁻ | 0.0646 |
| F⁻ | 0.0013 |
| H₃BO₃ | 0.0260 |

Natural seawater contains, besides these ions and compounds, trace elements such as lithium, neon, silicon, phosphorus, argon, titanium, vanadium, chromium, manganese, iron, cobalt, nickel, copper, zinc, arsenic, selenium, krypton, rubidium, molybdenum, silver, cadmium, antimony, iodine, cesium, tungsten, uranium and the like (Japan Meteorological Agency, “Marine Observation Guidelines”, 1990, p. 147).

Therefore, artificial seawater for culturing of algae is prepared by dissolving inorganic salts generating these ions in a specified ratio in water. A principal ingredient of the artificial seawater is sodium chloride and occupies about 60 to 70% by mass of inorganic salts to be added.

However, hardly soluble materials such as calcium sulfate, calcium carbonate and basic magnesium chloride are usually contained in sodium chloride and these materials cause an increase in the turbidity of seawater, which inhibits the transmission of light to thereby hinder the growth of algae or to be deposited onto the surface of algae to inhibit the growth of algae.

In order to prevent formation of such hardly soluble materials, attempts have been made to carry out a method of removing magnesium ions which are the source of the generation of basic magnesium chloride and calcium ions which are the source of the generation of calcium carbonate from an aqueous sodium chloride solution by adding chemicals such as soda ash. However, this method has a big problem that large-scale facilities are required and it takes a long time to treat a large volume of sodium chloride.

The fourth embodiment according to the present invention has been attained with the intention to provide a sodium chloride composition which never produce a hardly soluble material which is a cause of pollution of seawater and inhibition of growth of algae when the composition is used to prepare artificial seawater for culturing of algae.

### DISCLOSURE OF THE INVENTION

Usually, when a specified component is to be separated from a mixture solution by a chromatography, an adsorbent exhibiting selectivity to the component is used as a stationary phase while the mixture solution is used as a mobile phase and the mixture solution is passed through a column filled with the adsorbent to fractionate the passing fractions with time, thereby to collect a fraction containing the specified component as concentrated. As to the concentration of the specified component in the fraction at this time, the concentration never exceeds that of the component in the starting solution (hereinafter referred to as the initial concentration). The adsorbent continuedly adsorbs until the amount of the component reaches the adsorption amount determined by the adsorption capacity and the volume of the adsorbent. The concentration of the component in the solution gradually decreases, the amount of adsorption is saturated and the adsorbent loses the adsorbing ability finally, with the result that the concentration of the component in the passing solution becomes constant.

The present inventors have conducted extensive studies to remove a trace amount of impurity ions contained in an aqueous solution of a water-soluble compound efficiently from the solution and, as a result, found that, when, using an adsorption column filled with an adsorbent to which the same kind of ions as impurity ions are adsorbed or made to be adsorbed in advance, an aqueous solution to be treated is made to pass through the column, the phenomenon of abnormal adsorption chromatography occurs by which an eluate fraction having a higher impurity ion concentration than that of the starting solution is formed and then, when this eluate fraction is removed, an aqueous solution containing the high-purity water-soluble compound from which a trace amount of impurity ions are removed is obtained as a final processed solution after the starting solution is passed. Thus, the first embodiment of the present invention was completed based on this discovery.

Accordingly, the first embodiment of the present invention provides a method for removing trace impurity ions in a solution wherein, in carrying out removal of impurity ions by passing an aqueous solution of a water-soluble compound containing at least one kind of trace impurity ions through a column filled with an adsorbent capable of selectively adsorbing the impurity ions, a phenomenon of abnormal adsorption chromatography is caused so as to form an eluate fraction, in which the concentration of the trace impurity ions is higher than the concentration of the trace impurity ions in the starting solution, and the said fraction is discarded.

The present inventors focused their attentions onto sodium chloride as the water-soluble compound. The inventors have conducted extensive studies to remove potassium ions, magnesium ions and calcium ions as the impurities from the aqueous solution in an efficient manner and, as a result, found that when, using an adsorbent to which the same kind of ions as these ions are adsorbed in advance, an impurity ion-containing aqueous sodium chloride solution to be treated is made to pass through the column, the phenomenon of abnormal adsorption chromatography occurs by which an eluate fraction having a higher impurity ion concentration than that of the starting solution is formed, then, when this eluate fraction is removed, an aqueous solution fraction containing sodium chloride from which the impurity ions are removed is obtained as a final processed solution after the starting solution is passed and then sodium chloride crystals are crystallized, followed by the steps of a solid-liquid separation and drying, whereby high purity sodium chloride crystals can be separated and recovered. Thus, the second embodiment of the present invention was completed based on this discovery.

Accordingly, the second embodiment of the present invention provides a method for the preparation of purified sodium chloride crystals which comprises the steps of: (A) passing a concentrated aqueous solution of sodium chloride, of which the solid matter content is at least 50 g/liter, containing at least one kind of ions selected from potassium ions, magnesium ions and calcium ions as trace impurity ions through a column filled with an adsorbent exhibiting selective adsorptivity to at least one kind of ions selected from potassium ions, magnesium ions and calcium ions contained therein; (B) removing the trace impurity ions by utilizing the phenomenon of abnormal adsorption chromatography; then, (C) crystallizing sodium chloride from the thus treated solution followed by solid-liquid separation; and (D) drying the solid matter.

The present inventors have conducted various studies repeatedly to obtain sodium chloride reduced in the content of potassium ions and, as a result, sodium chloride crystals more remarkably reduced in the content of potassium ions than that of a commercially available conventional sodium chloride are obtained by bringing an aqueous solution of sodium chloride prepared from seawater, salt water, rock salt or crude salt into contact with an adsorbent having selective adsorptivity to potassium ions until the concentration of potassium ions is decreased to lower than a specified value and then by removing water. Thus, the third embodiment of the present invention was completed based on this discovery.

Accordingly, the third embodiment of the present invention provides a low potassium saline for medical use prepared by utilizing purified sodium chloride crystals of which the potassium ion concentration is lower than 0.07 mg/liter in a 20% by mass aqueous solution thereof and by dissolving the crystals in water and also provides a method for the preparation of an aqueous saline solution for medical use containing sodium chloride of which the concentration of potassium ions is lower than 0.07 mg/liter in a 20% by mass aqueous solution of sodium chloride, which method comprises a step of bringing an aqueous solution of sodium chloride containing potassium ions into contact with an adsorbent capable of selectively adsorbing potassium ions, a step of crystallization of sodium chloride crystals from an eluate solution having a decreased potassium ion concentration, a step of separation and drying of the thus crystallized sodium chloride crystals and a step of dissolving the purified crystalline sodium chloride obtained in this way in water.

On the other hand, the present inventors have conducted various studies as to artificial seawater for culturing of algae and, as a result, found that formation of hardly soluble materials is caused by the magnesium ions or, according to the case, calcium ions contained in the sodium chloride before the drying step among all steps of producing sodium chloride used as the base material and that, when the concentration of these ions is reduced to lower than a specified value, troubles caused by the hardly soluble materials can be limited. Thus, the fourth embodiment of the present invention was completed based on this discovery.

Accordingly, the fourth embodiment of the present invention provides a sodium chloride composition for the preparation of artificial seawater for culturing of algae which is prepared by utilizing low-magnesium sodium chloride crystals of which a magnesium ion concentration not to exceed 10 ppm in a 20% by mass aqueous solution thereof and by compounding the crystals with a necessary amount of inorganic ingredients for growth of algae and also provides, in carrying out preparation of the above sodium chloride composition from seawater, salt water or an aqueous solution of rock salt or crude salt (hereinafter referred to as a starting aqueous sodium chloride solution), a method for preparation of a sodium chloride composition for the preparation of artificial seawater for culturing of algae which comprises a step in which a starting aqueous sodium chloride solution containing magnesium ions or magnesium and calcium ions as impurity ions is passed through a column filled with an adsorbent capable of selectively adsorbing magnesium ions or magnesium and calcium ions so as to remove the magnesium ions or the magnesium and calcium ions from the aqueous solution to obtain an aqueous solution of sodium chloride of which the magnesium ion content and each of the magnesium ion content and the calcium ion content is decreased not to exceed 10 ppm in a 20% by mass aqueous solution thereof, a step of separating sodium chloride from the aqueous solution to obtain solid sodium chloride and a step of compounding the solid sodium chloride with inorganic nutrient ingredients necessary for growth of algae.

### BRIEF DESCRIPTION OF THE DRAWING

FIG. 1 is a graph showing the elution curve of potassium ion concentration obtained in Example 1.

FIG. 2 is a graph showing the elution curves of potassium ion, potassium ion and magnesium ion concentrations obtained in Example 2.

FIG. 3 is a graph showing the relationship between the ratio of the volume of the eluate to the volume of the column and the concentration of potassium ions in Example 3.

FIG. 4 is a graph showing the relationship between the ratio of the volume of the eluate to the volume of the column and the concentration of each impurity component in Example 4.

FIG. 5 is a graph showing the elution curve of potassium ion concentration for a 30% by mass aqueous sodium chloride solution in Example 5.

FIG. 6 is a graph showing the elution curves of magnesium ions and calcium ions in an aqueous sodium chloride solution in Reference Example 2.

### BEST MODE FOR CARRYING OUT THE INVENTION

Generally speaking, if the ions of the same kind as a trace impurity, namely, potassium ions are made to be adsorbed in advance on an adsorbent, e.g., an ammonium ion-type zeolite, which selectively adsorbs potassium ions, when an aqueous solution of a water-soluble compound containing at least one kind of trace impurity, for example, an aqueous sodium chloride solution containing a trace amount of potassium ions is passed through a column filled with the adsorbent, sodium ions as the principal component are replaced with the potassium ions present in the pores. The potassium ions with which the sodium ions have been replaced are eluted out together with potassium ions contained in the aqueous solution as an impurity, thereby to obtain a fraction containing the potassium ions in a concentration higher than the concentration of potassium ions initially contained in the aqueous solution. The mechanism of the occurrence of such a phenomenon of abnormal adsorption chromatography is still not well understood.

The first embodiment of the present invention relates to a method of obtaining a water-soluble compound improved in the purity by utilizing such a phenomenon of abnormal adsorption chromatography to remove trace impurity ions contained in an aqueous solution of the water-soluble compound. In the second to fourth embodiments of the present invention, trace impurity ions such as potassium ions, magnesium ions and calcium ions contained in an aqueous sodium chloride solution are removed by utilizing the above method to obtain sodium chloride crystals improved in the purity.

The phenomenon of abnormal adsorption chromatography is usually characterized by the presence of the following process steps. Specifically, when a column filled with an adsorbent containing the same kind of ions as the trace impurity to be removed in advance is used to carry out chromatography, the eluate fractions take the following sequences.

(1) A stage in which, after a concentrated solution of the desired material contain the trace impurities is introduced into the column, an eluate fraction made rich in the trace impurities is formed until the volume of the eluate from the column reaches five times of the column volume.

(2) A next stage in which eluate fractions of monotonously decreasing in the trace impurities are formed.

(3) A subsequent stage in which eluate fractions are formed in which the concentrations of the trace impurities are kept constant at a level lower than the initial concentrations (concentrations in the starting solution).

(4) A subsequent stage in which eluate fractions are formed in which the concentrations of the trace impurities are monotonously increased at a level lower than the initial concentrations.

(5) A stage, as the case may be, in which eluate fractions are formed in which the concentrations of the trace impurities are higher than the initial concentrations.

In the method of removing impurities according to the present invention, the phenomenon of abnormal adsorption chromatography lacking the step (1) among the above steps (1) to (5) (hereinafter referred to as pseudo phenomenon of abnormal adsorption chromatography) can also be utilized.

Examples of the aqueous solution of a water-soluble compound containing trace impurities for treatment in the method of the present invention may include aqueous reaction mixture solutions containing trace catalytic constituents obtained by a catalytic reaction and aqueous oligomer solutions containing trace monomers. Among these solutions, aqueous inorganic salt solutions containing trace impurities, for example, aqueous sodium sulfate solutions, aqueous sodium nitrate solutions and aqueous sodium phosphate solutions, particularly aqueous sodium halide solutions and especially an aqueous sodium chloride solution, containing corresponding potassium ions, lithium ions, magnesium ions and/or calcium ions as trace impurities are preferable.

The concentration of the aqueous solution of each water-soluble compound is usually selected from the range of concentration in which the lower limit is 1% by mass and the upper limit is within the saturation concentration and preferably in a range from 1 to 35% by mass though depending on the kind of the water-soluble compound, the types of the adsorbent to be used and conditions of the chromatography such as temperature, pressure and the rate of the solution to be fed to the column.

In order to obtain purified sodium chloride crystals in the second embodiment of the present invention, the concentration of the aqueous sodium chloride solution is selected from a range of, more preferably, 2 to 30% by mass but the solid content of the solution should usually be at least 50 g/liter.

In order to obtain the low potassium purified sodium chloride crystals used in saline for medical use according to the third embodiment of the present invention, the concentration of the aqueous sodium chloride solution is selected from a range of, more preferably, 3 to 30% by mass.

The concentrations of the trace impurities contained as the components other than the essential component in the aqueous solution of a water-soluble compound should desirably be not exceeding one twentieth and preferably not exceeding one fiftieth of the concentration of the principal water-soluble compound.

As to the adsorbent used in the method of the present invention, though it is dependent on the kinds of the trace impurities to be removed in the aqueous solution, cationic zeolites, e.g., ammonium ion-type zeolites and H⁺-type natural zeolites or cation- exchange resins are used when the impurities are metal ions and silica gels, aluminum oxide, activated carbons, cellulose, chemically modified silica gels, Sephadexes or polyacrylamide gels are used when the trace impurities are organic materials. These adsorbents are usually used in the form of particles having a particle diameter of 0.2 μm to 2.5 mm and preferably 0.1 to 2.5 mm.

When the impurities are potassium ions, magnesium ions or calcium ions, ion- exchange materials, e.g. ammonium ion-type zeolites, cellulosic ion exchangers and Sephadex ion exchangers which exhibit high adsorptivity to these ions are preferable. These materials are usually used in the form of particles having a particle diameter of 0.2 to 2.0 mm by introducing these particles into a chromatographic tube that is a column.

As the adsorbent which selectively adsorbs potassium ions in particular, positive ion-type natural zeolites, for example, a proton type, ammonium ion-type or alkylammonium ion-type clinoptilolite and mordenite are preferable. The above alkylammonium ion-type natural zeolite is obtained by carrying out positive-ion substitution of a natural zeolite with, for example, monomethylammonium, dimethylammonium, trimethylammonium or tetramethylammonium. Generally, these positive ion-type natural zeolites are respectively used in the form of a granular material having an average particle diameter of 0.2 to 500 μm by filling a column with the particles.

On the other hand, examples of the adsorbent to be used when removing magnesium ions or magnesium ions and calcium ions may include proton-type, ammonium ion-type or alkali metal ion-type natural zeolites or synthetic zeolites and chelating resins.

In the method of the present invention, it is necessary that the adsorbent which selectively adsorbs impurities has adsorbed ions of the same kinds as the impurities in advance. In the case of using, particularly, a positive ion-type natural zeolite as the adsorbent to remove potassium ions, it is advantageous to use an adsorbent which has adsorbed potassium ions in advance. The amount of these impurities to be adsorbed is selected from a range of 0.1 to 10 μmoles and preferably 1.0 to 10 μmoles per 1 g of the adsorbent. In this case, if an adsorbent to which the same kinds of ions as the impurities have been already adsorbed is used, it is unnecessary to carry out particular treatment for adsorption of the same kinds of ions as the impurities.

The linear rate when the aqueous solution of a water-soluble compound is made to flow through a column filled with the adsorbent is usually selected from a range of 0.1 to 300 cm/hr or, preferably, 0.1 to 100 cm/hr When the solution is fed, the passage may be promoted by changing pressure condition, namely by increasing or reducing the pressure as desired.

In order to properly carry out the method of removing impurities according to the present invention, all fractions of the column eluent of the aqueous solution of a water-soluble compound made to pass through the column are collected by fractionation to measure the concentrations of desired components in each fraction, thereby collecting fractions having similar concentrations. A solution in which a desired component is enriched can be obtained by collecting fractions in which a specified component is enriched altogether, and also, a high purity water-soluble compound can be obtained by collecting eluate fractions having a trace impurity concentration lower than the initial concentration.

It is further possible that a specified component adsorbed temporally on the column is desorbed out by using an adequate eluent to obtain a solution in which the component is concentrated.

A preferred embodiment of the method of the present invention will be described taking an aqueous solution containing a sodium salt in a concentration of 2.3% by mass and a trace amount of potassium salt as an example. When the above aqueous solution is made to pass through an adsorbent, for example, a positive ion-type zeolite, to which potassium ions have been adsorbed in advance, the potassium ions adsorbed on the adsorbent are eluted out into the mobile phase and a fraction in which the volume of the eluent has increased to reach five times the volume of the column (hereinafter referred to as the first fraction) has an impurity concentration higher than that in the starting solution. Thereafter, when the aqueous solution is continuously passed, the amount of trace impurities to be adsorbed to the adsorbent from the mobile phase is larger than the amount of the trace impurities to be desorbed from the adsorbent to the mobile phase whereby an eluate fraction in which the concentration of the impurity ions is lower than that in the starting solution appears. At this time, the concentration of the trace impurities varies in a range from 100 times or higher to 1/100 or lower of the concentration in the starting solution. When the concentration reaches the break point, it rises up to the concentration in the starting solution in the same manner as in the case of a break curve in the ordinary adsorption chromatography and there is the case where it becomes larger than the concentration in the starting solution according to the conditions.

The procedure in the method of the present invention can be performed in just the same way as in the case of ordinary adsorption chromatography. As to the conditions of the procedure, generally, the conditions of ambient temperature and atmospheric pressure are selected.

In the method of the present invention, actual occurrence of the phenomenon of abnormal adsorption chromatography can be confirmed by collecting each fraction of the column eluate separately and analyzing the respective fractions.

In the second embodiment of the present invention, from an aqueous solution of concentrated sodium chloride which contains at least one kind of ion selected from potassium ions, magnesium ions and calcium ions as trace impurities and has a solid content of 50 g/l or higher, purified sodium chloride crystals having an impurity content of 1% by mass or lower based on the weight of the solid can be obtained finally by utilizing the aforementioned method of removing impurity ions. Then, the adsorbent column on which impurity ions are adsorbed after the aqueous concentrated sodium chloride solution is made to flow therethrough may be regenerated by allowing an aqueous solution of, for example, hydrogen chloride or ammonium chloride for reusing. Also, at this time, if a group of fractions rich in the potassium ions, a group of fractions rich in the magnesium ions and a group of fractions rich in the potassium ions are respectively collected to carry out the above regenerating procedure, fractions rich in the potassium ions, magnesium ions and calcium ions respectively can be obtained. If these fractions are concentrated, a salt corresponding to each fraction, specifically, potassium chloride, magnesium chloride and calcium chloride can be recovered. Although no particular limitations are imposed on the concentration of the aqueous hydrogen chloride solution or aqueous ammonium chloride solution, it is preferably in the range from 0.5 to 5 M from the standpoint of handling easiness.

It is the most conventional method for obtaining sodium chloride crystals from an aqueous concentrated sodium chloride solution after removal of impurities by passing the solution through the column that the solution is evaporated to dryness. Besides the above method, water-soluble alcohols, for example, methyl alcohol, ethyl alcohol and propyl alcohol may be added to the processing solution to crystallize sodium chloride. Also, sodium hydroxide may be added to the processing solution to generate precipitates of impurities, which are then removed. Water-soluble alcohols may be added to the solution to crystallize sodium chloride to obtain higher purity sodium chloride.

For instance, fractions scanty in the potassium ions are collected and concentrated. Then, water-soluble alcohols (preferably ethyl alcohol) may be added to the concentrated fractions thereby to obtain sodium chloride crystals. When the solution is so rich in sodium chloride (20% by mass or richer), crystals can be obtained without carrying out any concentrating procedures. Specifically, sodium chloride crystals can be obtained in a high yield by adding a water-soluble alcohol (preferably ethyl alcohol) to the concentrated sodium chloride solution. Because the purity of sodium chloride is low under usual conditions, it is considered to occur that the purity is decreased by intermixing of impurities. However, because the starting aqueous solution is a high purity solution having a sodium chloride purity of 99.95% by mass or higher, almost no reduction is noted in the purity by intermixing of potassium, calcium and magnesium.

The crystallization method for high purity sodium chloride (sodium chloride purity: 99.99% by mass or higher) by the addition of a water-soluble alcohol is also effective in crystallization of high purity sodium chloride from a fraction from which magnesium ions have been removed after the treatment using a magnesium ion-selective ion exchanger and in crystallization of high purity sodium chloride (sodium chloride purity: 99.99% by mass or higher) from a fraction from which calcium ions have been removed after the treatment using a calcium ion-selective ion exchanger.

The method of the present invention can be conducted satisfactorily by collecting all of the column eluate fractions of concentrated aqueous sodium chloride solutions containing at least one kind of impurity ions among potassium, magnesium and calcium ions as impurity ingredients (content of evaporation residue to dryness 100 g/liter and sodium chloride purity in the evaporation residue 90% by mass or higher), determining the concentration of at least one kind of ions among potassium, magnesium and calcium ions, and combining those fractions having a similar concentration of at least one kind of ions among potassium, magnesium and calcium ions. By combining those fractions in which the ion concentration of potassium, magnesium and calcium ions is lower as compared with the initial concentration, a further enriched mother liquor of sodium chloride can be obtained.

The concentrated sodium chloride mother liquor obtained in this manner may be subjected to crystallization by evaporation crystallization or reaction crystallization to produce high purity sodium chloride (sodium chloride purity: 99.99% by mass or higher).

When a potassium ion-selective ion exchanger is used in the method of the present invention, ammonium ions or protons are suitable as the counter cations. The potassium ion-selective ion exchanger has high affinity to potassium ions and, therefore, potassium ions can be insufficiently removed only by hydrochloric acid treatment. Therefore, it is effective to carry out an ion exchange treatment (preferably, a treatment using an ammonium chloride solution) between potassium ions and ammonium ions having ionic radii close to that of potassium ions.

Though it is possible to use an ion exchanger made into an ammonium ion type directly for removal of potassium ions, there is a possibility of intermixing of ammonium chloride in the crystallization of sodium chloride. With regard to this matter, if the ion exchanger is made into a proton type, hydrochloric acid never separates and therefore it is ensured that high purity sodium chloride is obtained. It is however necessary to wash the ion exchanger sufficiently because hydrochloric acid treatment causes the adsorbent to dissolve so that the structural elements are easily eluted. This washing using hydrochloric acid is not limited to the above and is also effective to remove magnesium ions when a magnesium ion-selective ion exchanger is used or to remove calcium ions when a calcium ion-selective ion exchanger is used.

The low potassium purified sodium chloride crystals used for low sodium medical saline as the third embodiment of the present invention are characterized by the properties that the concentration of potassium ions is as low as to be lower than 0.07 mg/l and preferably 0.06 mg/l or lower in an aqueous solution containing these crystals in a concentration of 20% by mass. It is found that such a low potassium ion concentration is an unexpectedly low potassium ion concentration taking into consideration that, when commercially available conventional sodium chloride specified in Japanese Pharmacopoeia is dissolved in water to prepare a 20% by mass aqueous solution, the concentration of potassium ions in the solution is about 0.1 to 1.2 mg/l and, when salt obtained by evaporating commercially available conventional physiological saline to dryness is dissolved in water to prepare a 20% by mass aqueous solution, the concentration of potassium ions in the solution is about 0.3 mg/l.

When this concentration of potassium ions is converted into a relative amount based on the weight of the sodium chloride crystals, it is lower than 0.35 ppm.

In order to obtain such sodium chloride crystals having a low potassium ion content and constituting the medical salines of the present invention, for instance, potassium-containing sodium chloride obtained from seawater or salt water is first dissolved in water to prepare an aqueous solution. In this case, the concentration of sodium chloride in the aqueous solution is selected so as to be in a range usually from 1 to 35% by mass or, preferably, from 3 to 30% by mass though there are no particular limitations to the concentration.

As the adsorbent which is brought into contact with an aqueous solution of sodium chloride containing potassium ions to selectively adsorb potassium ions, positive ion-type natural zeolite, for example, a proton type, ammonium ion-type or alkylammonium ion-type clinoptilolite and mordenite are preferable. Among these materials, ammonium ion-type clinoptilolite is particularly preferable. The above alkylammonium ion-type natural zeolite is obtained by carrying out cation substitution of a natural zeolite with, for example, monomethylammonium, dimethylammonium, trimethylammonium or tetramethylammonium. Usually, these positive ion-type natural zeolites are used in the form of granules having an average particle diameter of 0.2 to 500 μm by filling a column therewith.

When the positive ion-type natural zeolite is used as the adsorbent to remove potassium ions, it is advantageous to remove the potassium ions by using the adsorbent to which potassium ions have been adsorbed in advance and utilizing the phenomenon of abnormal adsorption chromatography. At this time, the amount of the potassium ions to be adsorbed is preferably in a range from 0.1 to 10 μmoles per 1 g of the adsorbent.

If fractions made rich in the potassium ions are excluded by utilizing the phenomenon of abnormal adsorption chromatography, an aqueous sodium chloride solution containing potassium ions in a very low concentration can be obtained.

In a preferable procedure for producing medical salines obtained by dissolving the low-potassium-sodium chloride crystals of the present invention, the adsorbent particles capable of selectively adsorbing potassium ions are packed in a column having an appropriate size and an aqueous solution of potassium ion-containing sodium chloride is made to pass through the column, to collect the eluates from which sodium chloride crystals are crystallized. Although no particular limitation is imposed on the flow rate to be used at this time, a linear flow rate ranging usually from 0.1 to 300 cm/hr or, preferably, 0.1 to 100 cm/hr is used.

It is essential to continue liquid passing through the adsorbent-filled column until the potassium ion concentration in a 20% by mass aqueous solution of the sodium chloride crystals obtained thereby does not exceed 3.0 mg/l or, preferably does not exceed 1.5 mg/l or, more preferably, does not exceed 0.5 mg/l.

In order to separate sodium chloride crystals from the aqueous sodium chloride solution after passing through the column, it is the most usual way to evaporate the solution to dryness in the same manner as in the second embodiment of the present invention. Alternatively, water-soluble alcohols, for example, methyl alcohol, ethyl alcohol and propyl alcohol may be added to the processing solution to separate sodium chloride. Also, sodium hydroxide may be added to the processing solution to produce precipitates of impurities which are then removed and water-soluble alcohols are added to the resulting solution to crystallize sodium chloride thereby to obtain sodium chloride crystals having a still higher purity.

Although there can be a possible drawback that the purity of the whole solution is dropped by intermixing of other impurities in usual conditions because the purity of sodium chloride is low, a reduction in the purity is hardly caused by intermixing of other impurities if an aqueous solution of high purity sodium chloride is used as the starting solution. Also, the adsorbent column to which potassium ions have been adsorbed by passing the aqueous sodium chloride solution therethrough can be regenerated and reused by passing, for example, an aqueous solution of hydrogen chloride or ammonium chloride.

A low potassium medical saline prepared from low potassium-sodium chloride crystals having a potassium ion concentration lower than 0.07 mg/l and preferably 0.06 mg/l or lower when an aqueous solution containing these crystals in a concentration of 20% by mass and from water is obtained in this manner.

The medical saline obtained in this manner may be administered as such at a dose of about 1 to 2 g a day for inorganic salt supply agent when Na⁺ or Cl⁻ is deficient. Also, the medical saline may be used by preparing an aqueous solution containing it in a specified concentration as a diluent for injections, physiological solutions such as a Ringer solution and a Locke's solution and a sodium chloride injection. Besides the above, it may be used as a body fluid isotonic solution for washing of skins, wounded surfaces and mucosas, packings and washing of medical instruments.

The sodium chloride composition for preparation of artificial seawater for culturing of algae according to the fourth embodiment of the present invention is characterized by the use of sodium chloride preventive of the formation of hardly soluble materials, the sodium chloride being prepared by crystallizing sodium chloride crystals in which the concentration of magnesium ions and, as the case may be, calcium ions is a specified value or lower, followed by separation and drying of these crystals. It is necessary to use sodium chloride preventive of the formation of hardly soluble materials, the above sodium chloride being prepared by crystallizing sodium chloride crystals satisfying such requirements that the content of magnesium ions is 10 ppm or lower, namely, 0 to 10 ppm, and also, as the case may be, the content of calcium ions is 10 ppm or lower, namely 0 to 10 ppm calculated for an aqueous solution containing 20% by mass of these crystals, followed by separation and drying of the resulting crystals.

The above sodium chloride preventive of the formation of hardly soluble materials can be prepared as follows.

(1)A starting aqueous sodium chloride solution is passed through a column filled with an adsorbent exhibiting selective adsorptivity to magnesium ions or to magnesium ions and calcium ions and bearing the same kinds of ions as these ions adsorbed thereon beforehand to obtain an eluate solution which is subjected to crystallization of sodium chloride crystals to crystallize sodium chloride crystals containing magnesium ions or magnesium ions and calcium ions respectively in a decreased content followed by separation and drying thereof.

(2) The starting aqueous sodium chloride solution is subjected to evaporation concentration, addition of a non-solvent or addition of a precipitant thereby to crystallize sodium chloride crystals to be separated and the thus separated crystals are again converted into an aqueous solution, this procedure being repeated thereby to remove the impurities and to crystallize sodium chloride crystals containing a decreased amount of magnesium ions or magnesium and calcium ions followed by separation and drying thereof.

(3) Alternatively, magnesium ions or magnesium ions and calcium ions are removed from the starting aqueous sodium chloride solution by ion exchange treatment and then sodium chloride crystals decreased in the content of magnesium ions or magnesium ions and calcium ions are crystallized, followed by separation and drying of these crystals.

The method (1) is one which utilizes the method of the present invention for removing impurities, wherein the starting aqueous sodium chloride solution is passed through a column filled with the adsorbent which selectively adsorbs magnesium ions and, as the case may be, calcium ions, to remove magnesium ions or magnesium ions and calcium ions in the solution. Examples of the adsorbent used at this time may include proton type, ammonium ion-type or alkali metal ion-type natural zeolites and synthetic zeolites and chelate resins.

The starting aqueous sodium chloride solution is passed through a column filled with such an adsorbent to collect fractions reduced in magnesium ion concentration in the column eluate and then these fractions are either subjected to evaporation-crystallization to precipitate crystals, which are then separated and dried, or concentrated and then subjected to the so-called reactive crystallization performed by adding a non-solvent such as alcohols to precipitate crystals, which are then separated and dried, whereby desired sodium chloride crystals can be obtained.

In this case, if the magnesium ions-adsorbing capacity of the column is saturated and the concentration of magnesium ions in the column eluate reaches the concentration of magnesium ions of the solution to be added to the column, hydrochloric acid is passed through the column to cause desorption of the magnesium ions adsorbed on the column out of the column, whereby the column can be regenerated. This treatment for regeneration of the column by using hydrochloric acid can be performed irrespectively of the temperature; however, the preferable temperature for regeneration treatment is 20 to 80 ° C.

In the method (2), sodium chloride crystals reduced in the content of magnesium ions or magnesium ions and calcium ions are crystallized by the so-called regenerating method in which the crystallization of sodium chloride crystals is carried out repeatedly by evaporation-crystallization or reactive crystallization in the same manner as in the method (1), followed by separation and drying of these crystals.

Then, the method (3) is one in which magnesium ions and, as the case may be, calcium ions in the starting aqueous sodium chloride solution are exchanged with sodium ions or other alkali metal ions by ion exchange through a resin membrane obtained by introducing positive ion groups such as sulfonic acid groups, phosphonic acid groups, sulfate groups or phosphate groups into a polymer whose basic skeleton is a polyethylene, polystyrene, phenol resin or acrylic resin and, then, sodium chloride crystals are crystallized from the eluate solution, followed by separation and drying of these crystals.

These methods can be carried out either singly or in combinations of two or more.

The sodium chloride composition for preparation of artificial seawater for culturing of algae according to the present invention is produced by compounding the sodium chloride crystals preventive of the formation of hardly soluble materials as obtained in this manner with inorganic ingredients other than sodium chloride, for example, magnesium chloride, magnesium sulfate, calcium chloride, sodium sulfate, potassium chloride, potassium bromide, strontium chloride, sodium fluoride, sodium orthoborate, boric acid and sodium hydrogencarbonate, that are usually contained in natural seawater and are necessary for culturing of algae, and, further, as the case may be, trace components contained in natural seawater, for example, lithium, neon, silicon, phosphorus, argon, titanium, vanadium, chromium, manganese, iron, cobalt, nickel, copper, zinc, arsenic, selenium, krypton, rubidium, molybdenum, silver, cadmium, antimony, iodine, cesium, tungsten and uranium in a necessary amount.

Next, in order to prepare artificial seawater for culturing of algae by using the sodium chloride composition for preparation of artificial seawater for culturing of algae according to the present invention, the composition is dissolved in water such that the proportion of the weight of the sodium chloride crystals preventive of the formation of hardly soluble materials in the composition is 5 to 80 g or, preferably, 20 to 40 g based on 1 kg of water

When algae belonging to green algae, brown algae and red algae or, for example, a sea tangle, gulfweed, laver, glue plant, agar-agar, wakame seaweed, Gracilaria verrucosa and Chondrus ocellatus, are cultured in the artificial seawater prepared in this manner, these algae exhibit the same growth state as or, as the case may be, a better growth state than in the case of natural seawater.

In the following, the present invention is described in more detail by way of examples, which, however, are not intended to limit the present invention in any way.

### EXAMPLE 1

By using an apparatus system having a liquid-feeder pump, an adsorbent column and a fraction collector, potassium ions were separated from a 1 M aqueous sodium chloride solution containing, as impurities, 13 mg/l of potassium ions, 4 mg/l of magnesium ions and 2 mg/l of calcium ions.

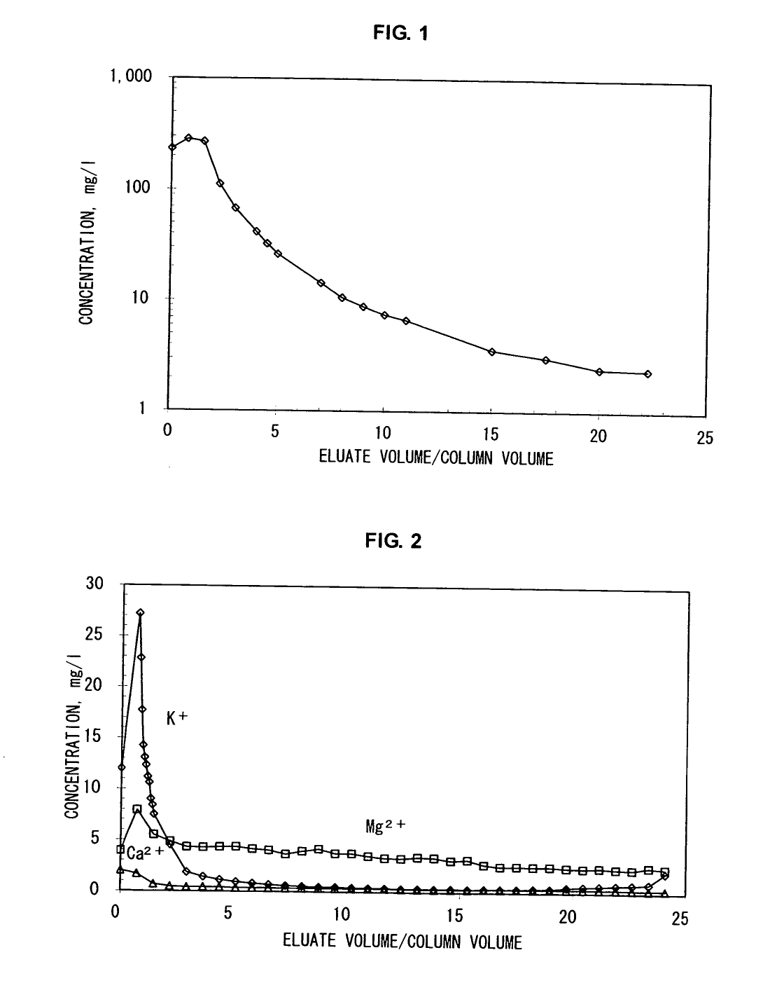

A glass-made column (inner diameter: about 10 mm, height: 500 mm) was filled with, as an adsorbent, an ammonium ion-type natural zeolite (trade name: “Sun Zeolite”, manufactured by Sun Zeolite Co., clinoptilolite, average particle diameter: 0.5 mm) to which about 5 μmol/g of potassium ions were adsorbed, up to a height of 470 mm, and the aforementioned aqueous sodium chloride solution was passed therethrough at a flow rate of 0.1 ml/min. in a thermostatted chamber (27° C.). The eluate from the column at this time was fractionally collected in 2-ml portions and the concentration of potassium ions in each fraction was analyzed quantitatively. An elution curve for the potassium ion concentration in each of the fractions is shown in FIG. 1. In this figure, the ordinate is for the potassium ion concentration (mg/l) of every 20th fraction and the abscissa is for the ratio of the volume of the eluate to the volume of the column.

As is clear from this diagram, fractions enriched in the potassium ions appear in the early stages followed by appearance of fractions depleted of potassium ions subsequently.

### EXAMPLE 2

By using an apparatus system having a liquid-feeder pump, an adsorbent column and a fraction collector, impurities were separated from a 1 M aqueous sodium chloride solution containing, as impurities, 13 mg/l of potassium ions, 4 mg/l of magnesium ions and 2 mg/l of calcium ions.

A glass-made column (inner diameter: 50 mm, height: 200 mm) was filled with, as an adsorbent, a H⁺ type natural zeolite (trade name: “Sun Zeolite”, manufactured by Sun Zeolite Co., clinoptilolite, average particle diameter: 0.5 mm) to which about 7 μmol/g of potassium ions were adsorbed, up to a height of 70 mm, and the aforementioned aqueous sodium chloride solution was passed therethrough at a flow rate of 1 ml/min. at 50° C.

Then, the eluate from the column was fractionally collected in 2-ml portions and each of the concentrations of potassium ions, magnesium ions and calcium ions in each fraction was analyzed quantitatively. The results are shown as a graph in FIG. 2.

In this figure, the ordinate is for the concentration (mg/l) of each impurity and the abscissa is for the ratio of the volume of the eluate to the volume of the column. It is understood from this figure that fractions enriched in the potassium ions and magnesium ions are eluted in the early stages and fractions from which potassium ions and magnesium ions have been removed are eluted subsequently. It is also noted that calcium ions are removed without being concentrated.

### EXAMPLE 3

Using an apparatus system having a liquid-feeder pump, an adsorbent column and a fraction collector, potassium ions were separated from an aqueous 1M-sodium chloride solution containing, as impurities, 13 mg/l of potassium ions, 4.5 mg/l of magnesium ions and 3.5 mg/l of calcium ions.

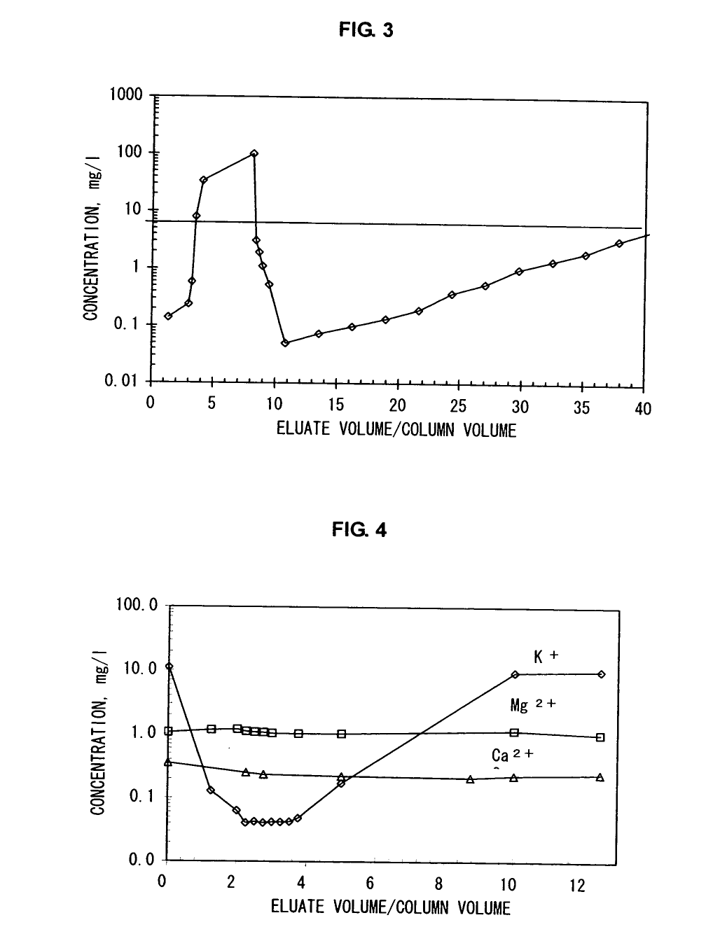

A glass-made column (inner diameter: 10 mm, height: 500 mm) was filled with, as an adsorbent, an ammonium ion-type natural zeolite (trade name: “Sun Zeolite”, manufactured by Sun Zeolite Co., clinoptilolite, average particle diameter: 0.5 mm) to which about 5 μmol/g of potassium ions was adsorbed, up to a height of 470 mm, and the aforementioned aqueous sodium chloride solution was passed therethrough at a linear flow rate of 30 cm/hr in a thermostatted chamber (27° C.). The relationship between the ratio of the volume of the eluate from the column to the volume of the column and the concentration of potassium ions (mg/l) in the eluate at this time was obtained. The concentration-elution curve obtained in this manner is shown in FIG. 3. It is understood from this figure that the phenomenon of abnormal adsorption chromatography took place and potassium ions were removed.

Next, fractions having a low potassium ion concentration were collected and concentrated by a rotary evaporator. Then, ethyl alcohol was added to the concentrated fractions to precipitate crystals, which were collected by filtration, washed with ethyl alcohol and dried to obtain purified sodium chloride.

The sodium chloride obtained in this manner was dissolved in water to prepare a 10% by mass aqueous solution, which was then analyzed by the atomic absorption spectrophotometric method to find that the concentration of potassium ions was 0.4 mg/l, the concentration of magnesium ions was 0.03 mg/l and the concentration of calcium ions was 1.3 mg/l.

### EXAMPLE 4

By using an apparatus system having a liquid-feeder pump, an adsorbent column and a fraction collector, impurities were removed from a 30% by mass aqueous sodium chloride solution containing, as impurities, 9 mg/l of potassium ions, 1 mg/l of magnesium ions and 0.8 mg/l of calcium ions.

A glass-made column (inner diameter: 10 mm, height: 500 mm) was filled with, as an adsorbent, an ammonium ion-type natural zeolite (trade name: “Octa Zeolite”, manufactured by Octa Zeolite Co., clinoptilolite, average particle diameter: 0.5 mm) up to a height of 450 mm and the aforementioned aqueous sodium chloride solution was passed therethrough at 50° C. at a linear flow rate of 9 cm/hr.

In this manner, the relationship between the ratio of the volume of the eluate from the column to the volume of the column and the concentration of each impurity ingredient (mg/l) was found. This relationship is shown as a graph in Fig. 4. It is understood from this figure that with regard to the potassium ions the significant phenomenon of abnormal adsorption chromatography took place and the concentration of the potassium ions was dropped to 0.05 mg/l.

Next, fractions having a low potassium ion concentration were collected and ethyl alcohol was added in a volume twice as large as that of the fractions to precipitate crystals, thus obtaining purified sodium chloride crystals.

The purified sodium chloride crystals obtained in this manner were, after vacuum drying, dissolved in water to prepare a 10% by mass aqueous solution, which was then analyzed by the atomic absorption spectrophotometric method to find that the concentration of potassium ions was 0.3 mg/l, the concentration of magnesium ions was 0.03 mg/l and the concentration of calcium ions was 0.04 mg/l.

### REFERENCE EXAMPLE 1

Six pharmacopoeial physiological saline products (NaCl concentration: 0.9% by mass) coming from different in production lots were each evaporated to dryness to obtain solid matters which were then used to prepare six 20% by mass aqueous sodium chloride solutions. Separately, with regard to sodium chloride (crytals) products of (A and B) pharmacopoeia coming from two different in producers, six products of different production lots in each of the products A and B, specifically, a total of twelve products were obtained and each of them was made into a 20% by mass aqueous solution. The content of potassium ions in each of the above total 18 types of aqueous solutions was determined by the atomic absorption spectrophotometric method. The results are shown in Table 2.

TABLE 2

| | Potassium ion content (mg/liter) | | |
|---|---|---|---|
| Sample | Pharmacopoeial | Pharmacopoeial | Pharmacopoeial |
| No. | physiological saline | sodium chloride A | sodium chloride B |
| 1 | 0.25 | 0.11 | 1.07 |
| 2 | 0.29 | 0.10 | 1.50 |
| 3 | 0.31 | 0.09 | 1.15 |
| 4 | 0.21 | 0.09 | 1.09 |
| 5 | 0.45 | 0.10 | 1.20 |
| 6 | 0.30 | 0.11 | 1.20 |
| Mean | 0.30 | 0.10 | 1.20 |

### EXAMPLE 5

By using an apparatus system having a liquid-feeder pump, an adsorbent column and a fraction collector, potassium ions were removed from a 30% by mass aqueous sodium chloride solution containing, as an impurity, 9 mg/l of potassium ions.

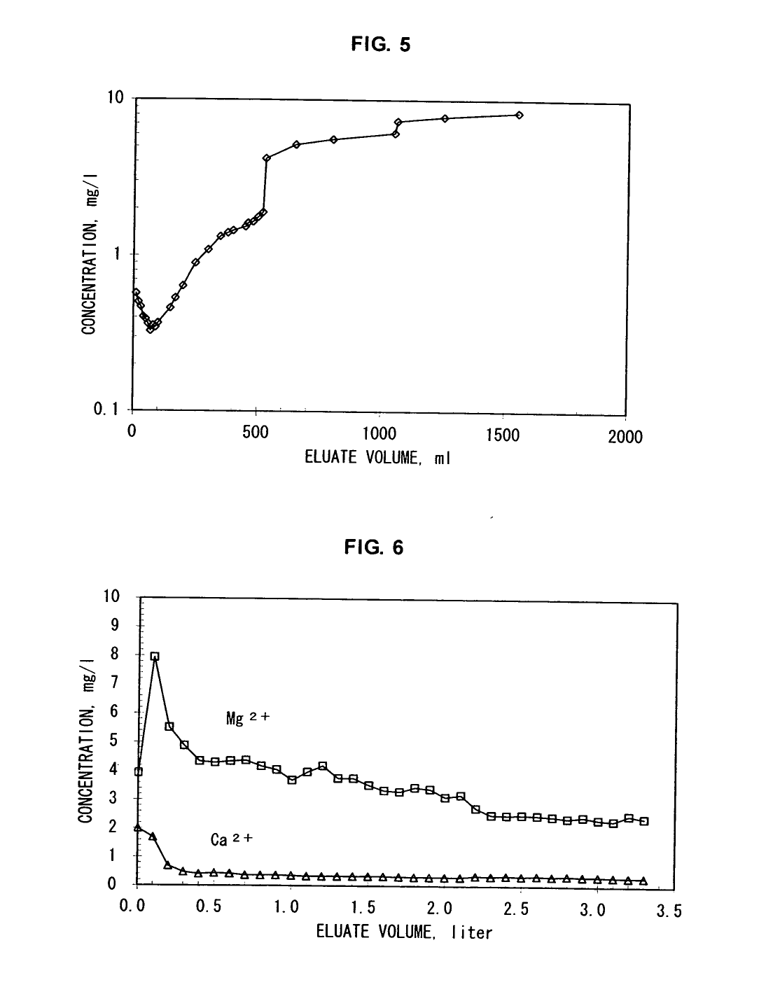

A glass-made column (inner diameter: 10 mm, height: 500 mm) was filled with, as an adsorbent, an ammonium ion-type natural zeolite (trade name: “Sun Zeolite”, manufactured by Sun Zeolite Co., clinoptilolite, average particle diameter: 0.5 mm) to which about 5 μmol/g of potassium ions were adsorbed, up to a height of 450 mm, and the aforementioned aqueous sodium chloride solution was passed therethrough at a flow rate of 20 ml/hr in a thermostatted chamber (27° C.). The elution curve of the potassium ions in this case is shown in FIG. 5.

In this figure, the ordinate is for the concentration (mg/l) of the potassium ions and the abscissa is for the volume of the eluate (ml). As is understood from this figure, potassium ions were decreased in the early stage and the concentration of the potassium ions was dropped to about 0.4 mg/l and then raised. The eluted fractions (fractions corresponding to an eluate volume of 30 ml to 400 ml in FIG. 5) in which the concentration of potassium ions in a 30% by mass aqueous sodium chloride solution was 1.5 mg/l or lower were collected. Ethyl alcohol was added to the collected fractions to obtain low potassium-sodium chloride crystals.

Next, an aqueous ammonium chloride solution having a concentration of 3 mol/l was passed through the column at 20° C. to regenerate the adsorbent.

The above procedure of collecting fractions in which the concentration of the potassium ions in the 30% by mass aqueous sodium chloride solution was 1.5 mg/l or lower, the procedure of crystallizing sodium chloride from this collected fractions and the procedure of regenerating the column were respectively repeated six times, to obtain six lots of low potassium-sodium chloride crystals. The content of potassium ions in the low potassium-sodium chloride crystals was quantitatively determined by the atomic absorption spectrophotometry in the same manner as in Reference Example 1. The results are shown in Table 3.

TABLE 3

| | Potassium ion content in 20% by mass |
|---|---|
| Sample No. | aqueous sodium chloride solution (mg/liter) |
| 1 | 0.05 |
| 2 | 0.05 |
| 3 | 0.06 |
| 4 | 0.05 |
| 5 | 0.06 |
| 6 | 0.05 |
| Mean | 0.053 |

As mentioned above, the contents of potassium ions in the sodium chloride that form the material of the present invention are lower than that of any one of the commercially available products shown in Reference Example 1.

### COMPARATIVE EXAMPLE

Potassium ions in a 30% by mass aqueous sodium chloride solution were removed in an evaporation crystallization step. Sodium chloride having a potassium ion concentration of 5.5 mg/l in a 30% by mass aqueous sodium chloride solution was used as a sample to be subjected to the evaporation crystallixation. The crystallization was carried out using six types of evaporation crystallization samples which were different from each other to obtain sodium chloride crystals by a step in six lots. The results are shown in Table 4

TABLE 4

| Sample | Potassium ion content in 20% by mass |
|---|---|
| No. | aqueous sodium chloride solution (mg/liter) |
| 1 | 0.96 |
| 2 | 1.02 |
| 3 | 1.32 |
| 4 | 0.96 |
| 5 | 0.72 |
| 6 | 0.90 |
| Mean | 0.98 |

As is understood from the above table, the content of potassium ions in the above sodium chloride obtained without using the method of the present invention was about the same as or higher than those of the commercially available products shown in Reference Example 1.

### REFERENCE EXAMPLE 2

By using an apparatus system having a liquid-feeder pump, an adsorbent column and a fraction collector, magnesium ions and calcium ions were removed from a 1M aqueous sodium chloride solution prepared by dissolving rock salt in distilled water. The concentration of the magnesium ions and the concentration of the calcium ions in the starting aqueous solution were 4.4 mg/l and 5.2 mg/l, respectively.

A glass-made column (inner diameter: 50 mm, height: 200 mm) was filled with, as an adsorbent, a proton type natural zeolite (trade name: “Sun Zeolite”, manufactured by Sun Zeolite Co., clinoptilolite, average particle diameter: 0.5 mm) up to a height of 70 mm, and the aforementioned aqueous sodium chloride solution was passed therethrough at a flow rate by volume of 60 ml/hr in a thermostatted chamber (27° C.). Elution curves are shown in FIG. 6 for the magnesium ions and calcium ions.

In this figure, the ordinate is for the concentration of each species of ions (mg/l) and the abscissa is for the volume of the eluate (liters). It is understood from this figure that the magnesium ions were concentrated in the early stage and then magnesium ions were removed subsequently while calcium ions were removed without being first concentrated.

In the next place, the eluate fractions (eluate fractions corresponding to the eluate volume of 2.3 to 3.3 liters in FIG. 6) in which the concentration of magnesium ions in 1M sodium chloride was 2.5 ppm or lower (the concentration of magnesium ions in a 20% by mass aqueous sodium chloride was 8.6 ppm or lower) were collected. Ethyl alcohol was added to the collected fractions to obtain low-magnesium, low-calcium sodium chloride crystals.

The above procedure of collecting the eluate fractions in which the concentration of magnesium ions in 1M sodium chloride was 2.5 ppm or lower (the concentration of magnesium ions in a 20% by mass aqueous sodium chloride solution was 8.6 ppm or lower) were collected, the precipitation (crystallization) procedure and the procedure of regenerating the column were each repeated six times, to obtain six lots of low-magnesium, low-calcium sodium chloride. The content of magnesium ions in the six lots of sodium chloride was quantitatively determined by the atomic absorption spectrophotometry for the low-magnesium, low-calcium sodium chloride crystals as dried at 50° C. for 16 hours in a vacuum drier and returned to an aqueous solution by dissolving the dried product in distilled water. The mean content of magnesium in the six lots of the low-magnesium, low-calcium sodium chloride crystals was 0.00015% by mass with a coefficient of variation CV (relative standard deviation) of 36.5. The coefficient of variation CV (relative standard deviation) was calculated by multiplying the value obtained by dividing the standard deviation S (S=σ₋₁) for the samples (specimens) by the mean X of the sample (specimen), with 100. Further, the untreated sodium chloride, namely the sodium chloride before the treatment for removal of magnesium ions and calcium ions, was subjected to the determination of the contents of magnesium ions in the six lots of untreated sodium chloride to obtain a mean value of 0.00753% by mass with a coefficient of variation CV (relative standard deviation) of 7.59. The results are shown in Table 5.

TABLE 5

Magnesium content (% by mass)

| | Low-magnesium, low- | Untreated sodium |
|---|---|---|
| Sample No. | calcium sodium chloride | chloride |
| 1 | 0.00010 | 0.00753 |
| 2 | 0.00020 | 0.00741 |
| 3 | 0.00020 | 0.00766 |
| 4 | 0.00010 | 0.00817 |
| 5 | 0.00010 | 0.00651 |
| 6 | 0.00020 | 0.00792 |
| Mean | 0.00015 | 0.00753 |
| Coefficient | 36.5 | 7.59 |
| of variation | | |

The measured values of the contents of magnesium in the low-magnesium, low-calcium sodium chloride and in the untreated sodium chloride were statistically treated to make the homoscedastic test and the T-test. As a result of the homoscedastic test, the homoscedastic properties of the population of low-magnesium, low-calcium sodium chloride and untreated sodium chloride could be supposed (F=4.435, probability of significance p=0.061>level of significance α=0.0050). As a result of the T-test (test as to a difference between two population means), t-value=−31.499 and probability of significance (both sides) p<0.01. The content of magnesium in the untreated sodium chloride on the mean was 0.00753% by mass whereas the content of magnesium in the low-magnesium, low-calcium sodium chloride on the mean was 0.00015% by mass. It is understood from the content of magnesium in the low-magnesium, low-calcium sodium chloride, magnesium was significantly (p<0.01) removed from the untreated sodium chloride.

Low-magnesium, low-calcium sodium chloride obtained in this manner was dried at 110° C. to prepare sodium chloride preventive of the formation of hardly soluble materials.

### EXAMPLE 6

A sodium chloride composition for preparation of 25 liter artificial seawater for culturing of algae was prepared by admixing 548 g of the sodium chloride preventive of the formation of hardly soluble materials as obtained in Reference Example 2 with 250 g of magnesium chloride hexahydrate, 92.5 g of sodium sulfate, 35.0 g of calcium chloride dihydrate, 15.8 g of potassium chloride, 4.5 g of sodium hydrogen carbonate, 2.25 g of potassium bromide, 0.75 g of orthoboric acid, 0.25 g of strontium chloride, 0.13 mg of iron (III) chloride hexahydrate, 8.75 mg of sodium glycerophosphate pentahydrate and 4.0 mg of sodium nitrate. Separately, for comparison, untreated sodium chloride crystals were crystallized, followed by separation and drying to prepare sodium chloride without the treatment for preventing formation of hardly soluble materials, which was then used to prepare a comparative sodium chloride composition for the preparation of artificial seawater for culturing of algae. Next, the composition was sealed in a laminate bag, which was then stored in a 20° C. thermostatted chamber. In the above manner, each six lots of sodium chloride compositions for the preparation of artificial seawater for culturing of algae were prepared by using sodium chlorides preventive of the formation of hardly soluble materials prepared by crystallizing six lots of low-magnesium, low-calcium sodium chloride crystals, followed by separation and drying and sodium chlorides without treatment for preventing formation of hardly soluble materials prepared by crystallizing six lots of untreated sodium chloride crystals, followed by separation and drying. Those compositions were stored for 30 days after prepared and then each bag was opened to measure turbidity. The measurement of turbidity was made as follows: 40 g of sodium chloride composition for the preparation of artificial seawater for culturing of algae was dissolved in 1 liter (30° C.) of distilled water, which was then stirred for 5 minutes, and was allowed to stand for 1 minute and then poured into 50 mm quartz cells in lots to measure absorbance at a wavelength of 660 nm by using a spectrophotometer UV-120-01 manufactured by Shimadzu Corporation.

The mean value of the absorbance of 4% by mass aqueous solutions of the sodium chloride compositions for the preparation of artificial seawater for culturing of algae, which compositions were produced by using the above sodium chloride crystals preventive of the formation of hardly soluble materials prepared by crystallizing the six lots of the low-magnesium, low-calcium sodium chloride crystals, followed by separation and drying, was 0.0005 with a coefficient of variation CV (relative standard deviation) of 109.5.

The coefficient of variation CV (relative standard deviation) was calculated by multiplying the value obtained by dividing the standard deviation S (S=σₙ₋₁) of the samples (specimens) by the mean X of the samples (specimens), with 100. Also, the mean absorbance of 4% by mass aqueous solutions of the sodium chloride compositions for the preparation of artificial seawater for culturing of algae, which compositions were produced by using sodium chloride without the treatment for preventing the formation of hardly soluble materials prepared by crystallizing the six lots of untreated sodium chloride crystals, followed by separation and drying, was 0.0028 with a coefficient of variation CV (relative standard deviation) of 26.6. A homoscedastic test and a t test were performed after statistics processing of the measured values of turbidity of the 4% by mass aqueous solutions of the sodium chloride compositions used in preparation of artificial seawater for culturing of algae by using sodium chlorides preventive of the formation of hardly soluble materials as prepared by crystallizing the low-magnesium, low-calcium sodium chloride crystals, followed by separation and drying and sodium chlorides without the treatment for preventing the formation of hardly soluble materials prepared by crystallizing the untreated sodium chloride crystals, followed by separation and drying. As a result of the homoscedastic test, the homoscedastic properties could be supposed (F=0.094, probability of significance p=0.765 >level of significance α=0.050) in relation to the populations of the above sodium chloride preventive of the formation of hardly soluble materials as prepared by crystallizing low-magnesium, low-calcium sodium chloride crystals, followed by separation and drying and the above sodium chloride without the treatment for preventing the formation of hardly soluble materials prepared by crystallizing untreated sodium chloride crystals, followed by separation and drying. The t test (test for finding a difference in means of two populations) gave the result of t-value=−6.139 and probability of significance (both sides) p<0.01. The mean turbidity of 4% by mass aqueous solutions of the sodium chloride composition for the preparation of artificial seawater for culturing of algae using the sodium chloride without the treatment for preventing the formation of hardly soluble materials prepared by crystallizing the untreated sodium chloride crystals, followed by separation and drying was 0028. On the other hand, the mean turbidity of 4% by mass aqueous solutions of the sodium chloride composition for the preparation of artificial seawater for culturing of algae using sodium chloride preventive of the formation of hardly soluble materials prepared by crystallizing low-magnesium, low- calcium sodium chloride crystals, followed by separation and drying was 0.0005, to find that the aqueous solution was significantly (p<0.01) decreased in the turbidity as compared with the 4% by mass aqueous solution of the sodium chloride composition for the preparation of artificial seawater for culturing of algae using the sodium chloride without the treatment for preventing the formation of hardly soluble materials prepared by crystallizing untreated sodium chloride crystals, followed by separation and drying. These results are shown in Table 6.

TABLE 6

| | Turbidity (A 660 nm) | |
|---|---|---|
| | A 4% by mass aqueous | A 4% by mass aqueous |
| | solution of a sodium chloride | solution of a sodium |
| | composition used in | chloride composition used |
| | preparation of an artificial | in preparation of an |
| | seawater for culturing of | artificial seawater for |
| | algae by using sodium | culturing of algae by using |
| | chloride preventive of the | sodium chloride without |
| | formation of hardly soluble | treatment of preventing |
| | materials as prepared by | formation of hardly soluble |
| | crystallization of low- | materials as prepared by |
| | magnesium, low-calcium | crystallization of untreated |
| | sodium chloride followed by | sodium chloride followed |
| Sample No. | separation and drying | by separation and drying |
| 1 | 0.000 | 0.002 |
| 2 | 0.001 | 0.004 |
| 3 | 0.001 | 0.003 |
| 4 | 0.001 | 0.003 |
| 5 | 0.000 | 0.002 |
| 6 | 0.000 | 0.003 |
| Mean | 0.0005 | 0.0028 |
| Coefficient | 109.5 | 26.6 |
| of variation | | |

### APPLICATION EXAMPLE

By using a 3.5% by mass aqueous solution of the sodium chloride composition used in preparation of an artificial seawater for growing of algae obtained in Example 6 by using sodium chloride preventive of the formation of hardly soluble materials as prepared by crystallization of low-magnesium, low-calcium sodium chloride crystals followed by separation and drying (hereinafter referred to as “artificial seawater of the present invention”) and a 3.5% by mass aqueous solution of a sodium chloride composition used in preparation of an artificial seawater for growing of algae by using sodium chloride without treatment of preventing formation of hardly soluble materials as prepared by crystallization of untreated sodium chloride followed by separation and drying (hereinafter referred to as “artificial seawater for control”), an algae growing experiment was performed.

Specifically, the growing ends (called apical fragments) 5 mm in length were cut out from a unialga culture strain of sea alga “Gracilariaceae Gracilaria chorda” belonging to Red algae, Gracilaria and six apical fragments per flask were added to a conical flask containing 200 ml of the artificial seawater. Culturing conditions were set as follows: temperature: 20° C., light intensity: 60 μmol/cm² s and lighting cycles: 14 hours bright period and 10 hours dark periods. Artificial seawater which was a culture solution was refreshed every week and the flask was stirred at a rate of 100 rpm during culturing. Each of the number of samples used for experiments using the artificial seawater of the present invention and the number of samples used for experiments using the artificial seawater for control was set to 5. The wet mass of the marine alga grown in each artificial seawater after the marine alga was cultured for 4 weeks was measured.

The mean wet mass of the marine algae grown in the artificial water of the present invention was 62.6 mg and the coefficient of variation CV (relative standard deviation) was 7.45. On the other hand, the mean wet mass of the marine algae grown in the artificial water for control was 52.1 mg and the coefficient of variation CV (relative standard deviation) was 9.64. A homoscedastic test and a t test were performed after statistics processing of the measured values of the wet mass of the marine algae grown in the artificial seawater of the present invention and the artificial seawater for control. As a result of the homoscedastic test, the homoscedastic properties of the populations of the above artificial seawater of the present invention and the artificial seawater for control could be supposed (F=0.008, probability of significance p=0.929 >level of significance α=0.050). The t test (test for finding a difference in means of two populations) gave the result of t-value=3.438 and probability of significance (both sides) p<0.01. The mean of the wet mass of the marine algae grown in the artificial for control was 52.1 mg. On the other hand, the mean of the wet weight of the marine algae grown in the artificial seawater of the present invention was 62.6 mg to find that the amount of the marine alga (wet mass) which could be cultured was increased significantly (p <0.01) as compared with the wet mass of the marine alga grown in the artificial seawater for control. The results are shown in Table 7.

TABLE 7

Wet mass of marine alga (mg)

| Sample | Artificial seawater of | Artificial seawater |
|---|---|---|
| No. | the present invention | for control |
| 1 | 60.1 | 52.4 |
| 2 | 58.1 | 43.6 |
| 3 | 60.8 | 55.0 |
| 4 | 64.2 | 53.0 |
| 5 | 70.0 | 56.5 |
| Mean | 62.6 | 52.1 |
| Coefficient | 7.45 | 9.64 |
| of variation | | |

### INDUSTRIAL APPLICABILITY

According to the first embodiment of the present invention, an aqueous solution of water-soluble compounds including trace impurities is introduced into a column filled with an adsorbent capable of selectively adsorbing these trace impurities, making it possible to remove these trace impurities efficiently by utilizing the phenomenon of abnormal adsorption chromatography. Moreover, trace components can be concentrated when the adsorbent is regenerated and also, the adsorbent can be used repeatedly and this embodiment is therefore industrially advantageous.

According to the second embodiment of the present invention, high purity sodium chloride crystals can be obtained at low costs from a concentrated aqueous sodium chloride solution containing at least one kind of impurities selected from potassium ions, and magnesium ions by utilizing the phenomenon of abnormal adsorption chromatography and therefore, the present invention has a high utilizability value in industrial fields requiring such high purity sodium chloride.

According to the third embodiment of the present invention, a medical saline is provided as prepared from sodium chloride crystals containing potassium ions in a greatly decreased content as compared with conventional sodium chloride-based medical materials and water so that an advantage is obtained that onset of hyperkalemia can be prevented leading to a very great utilizability value of the present invention in the pharmaceutical industries.

According to the fourth embodiment of the present invention, a sodium chloride composition for the preparation of artificial seawater for culturing of algae is provided, the composition providing artificial seawater for culturing of algae having reduced turbidity when it is prepared as an aqueous solution, free from a rise in the turbidity of the aqueous solution even if it is stored for a prolonged time and is reduced in the risk of growth inhibition caused by the adsorption of hardly soluble materials onto the surface of algae, showing that the present invention has a very great utilizability value in marine products culturing industries.

What is claimed is:

1. A method for removing trace impurity ions in a solution wherein, in carrying out removal of impurity ions by passing an aqueous solution of a water-soluble compound containing at least one kind of trace impurity ions through a column filled with an adsorbent capable of selectively adsorbing the impurity ions, an abnormal adsorption-chromatographic phenomenon is caused so as to form an eluate fraction, in which the concentration of the trace impurity ions is higher than the concentration of the trace impurity ions in the starting solution, and the said fraction is discarded.

2. The method for removing trace impurity ions described in claim 1 in which the concentration of the trace impurity ions in the aqueous solution does not exceed one-twentieth of the concentration of the water-soluble compound as the principal ingredient.

3. The method for removing trace impurity ions described in claim 1 in which the water-soluble compound is an inorganic salt.

4. A method for the preparation of purified sodium chloride crystals characterized by comprising the steps in which a concentrated aqueous solution of sodium chloride, of which the solid matter content is at least 50 g/liter, containing at least one kind of ions selected from potassium ions, magnesium ions and calcium ions as trace impurity ions is passed through a column filled with an adsorbent exhibiting selective adsorptivity to at least one kind of ions selected from potassium ions, magnesium ions and calcium ions contained therein as the step (A), the trace impurity ions are removed by utilizing the phenomenon of abnormal adsorption-chromatography as the step (B), then sodium chloride is crystallized from the thus treated solution followed by solid-liquid separation as the step (C) and the solid matter is dried as the step (D).

5. The method for the preparation of purified sodium chloride crystals described in claim 4 in which the concentration of the impurity ions in the concentrated aqueous sodium chloride solution does not exceed 1% by mass relative to the total content of the solid matter in the said aqueous solution.

6. The method for the preparation of purified sodium chloride crystals described in claim 4 to which an additional step is added in which an aqueous solution of hydrogen chloride or ammonium chloride is passed through the column filled with the adsorbent after passing of the concentrated aqueous sodium chloride solution in step (A) so as to regenerate the adsorbent.

7. The method for the preparation of purified sodium chloride crystals described in claim 4 in which crystallization of sodium chloride in step (C) is carried out with addition of a water-soluble alcohol to the treatment solution.

8. The method for the preparation of purified sodium chloride crystals described in claim 7 in which the addition of a water-soluble alcohol to the treatment solution in crystallization of sodium chloride in step (C) is preceded by the formation of precipitates of impurity ions with addition of sodium hydroxide to the treatment solution.

9. An aqueous saline solution for medical use prepared by utilizing the purified crystalline sodium chloride which is the sodium chloride obtained by the method of either one of claims 4 to 8 of which the potassium ion concentration is lower than 0.07 mg/liter in an aqueous solution in a concentration of 20% by mass and by dissolving the same in water.

10. A method for the preparation of an aqueous saline solution for medical use containing sodium chloride of which the concentration of potassium ions is lower than 0.07 mg/liter in a 20% by mass aqueous solution of sodium chloride, which method comprises a step of bringing an aqueous solution of sodium chloride containing potassium ions into contact with an adsorbent capable of selectively adsorbing potassium ions, a step of crystallization of sodium chloride crystals from an eluate solution having a decreased potassium ion concentration, a step of separation and drying of the thus crystallized sodium chloride crystals and a step of dissolving the purified sodium chloride crystals obtained in this way in water.

11. The method for the preparation of an aqueous saline solution for medical use described in claim 10 in which the adsorbent capable of selectively adsorbing potassium ions is a cation-type natural zeolite.

12. The method for the preparation of an aqueous saline solution for medical use described in claim 11 in which the cation-type natural zeolite is an ammonium ion-type chrinoptirolite.

13. A sodium chloride composition for preparation of artificial seawater for culturing of algae formed by using a low-magnesium sodium chloride crystals which is the sodium chloride crystals obtained by the method of either one of claims 4 to 8 giving a 20% by mass aqueous solution containing magnesium ions in a concentration not exceeding 10 ppm and by compounding the same with inorganic ingredients necessary for growth of algae.

14. A sodium chloride composition for preparation of artificial seawater for culturing for algae formed by using a low-magnesium, low-calcium crystalline sodium chloride which is the sodium chloride crystals obtained by the method of either one of claims 4 to 8 giving a 20% by mass aqueous solution containing magnesium ions in a concentration not exceeding 10 ppm and calcium ions in a concentration not exceeding 10 ppm, and by compounding the same with inorganic ingredients necessary for growth of algae.

15. A method for the preparation of a sodium chloride composition for preparation of artificial seawater for culturing of algae which comprises a step in which an aqueous solution of starting sodium chloride containing magnesium ions or magnesium and calcium ions as impurity ions is passed through a column filled with an adsorbent capable of selectively adsorbing magnesium ions or magnesium and calcium ions so as to remove the magnesium ions or magnesium and calcium ions from the said aqueous solution to obtain an aqueous solution of sodium chloride of which the magnesium ion content or each of the magnesium ion content and the calcium ion content is decreased not to exceed 10 ppm in a 20% by mass aqueous solution thereof, a step for obtaining solid sodium chloride by separating sodium chloride from the said aqueous solution and a step of compounding the said solid sodium chloride with inorganic nutrient ingredients necessary for growth of algae.
